(19) United States
(12) Patent Application Publication
(10) Pub. No.: US 2002/0032449 A1
(43) Pub. Date: Mar. 14, 2002

(54) Device for recording craniofacial data with reference to a fixed plane

(76) Inventors: Monica Rota, Bergamo (IT); Giovanni Scotti, Bergamo (IT)

Correspondence Address: MODIANO & ASSOCIATI, Via Meravigli, 16, MILANO, 20123, IT

(21) Appl. No.: 09/947,397

(22) Filed: Sep. 7, 2001

(30) Foreign Application Priority Data
Sep. 13, 2000 (IT) MI2000A001995

(51) Int. Cl. A61B 17/56

(52) U.S. Cl. 606/102

(57) ABSTRACT

A device for recording craniofacial data, particularly for determining facial asymmetries, comprising a plate-like element to which a supporting element for positioning the device in the mouth of a patient is connected in a fixed manner, the plate-like element being provided with a plurality of arms whose position is adjustable, the arms being suitable to support measurement means which are arranged on a vertical plane with respect to the plane on which the plate-like element is formed, means for defining a reference plane being provided in order to refer the measurements recorded by the measurement means to the reference plane.

### BACKGROUND OF THE INVENTION

The present invention relates to a device for recording craniofacial data with reference to a fixed plane. More particularly, the invention relates to a gnatho-cranial-postural measurement device for recording and transferring individual craniofacial data with respect to a fixed plane.

It is known that the facebows currently used to record and subsequently position in an articulator the upper maxillary arch use, as their fundamental references, the acoustic meatuses and a single right or left infraorbital anterior point (Frankfort planes) or, as an alternative, the acoustic meatuses and the anterior point related to the nasal spine (Camper's plane).

These facebows, during recording, are supported and find their stability and position by means of a nasal support and record, by means of an articulated bite fork, the spatial position of the upper dental arch.

The only reference of the recordings made is therefore constituted by three points of the cranium of the subject (which can move in space), but such recordings have no reference to a universal plane or fixed reference.

In practice, the facebows are characterized by two lateral arms of equal length which are rigidly coupled or able to slide symmetrically in a front cross-member. At the center of the cross-member there is an articulation which supports, and allows to orientate and position in three dimensions, the bite fork for recording the upper dental arch.

Clearly, these recording methods completely ignore the variables related to asymmetries on the horizontal and frontal planes, as well as the random but morphological combination of asymmetries on the sagittal, horizontal and frontal planes.

Essentially, the recorded data, indeed because they have a single anterior reference point, do not take into account the possible physiological asymmetries that are typical of each individual, and since they are not referred to a universal fixed plane (but only to craniofacial points of the subject), they are applied to a fixed axis which is dictated by the facebow and by the articulator being used.

The need is felt to find reference planes which are fixed, specific and comparable over time in order to record defects in prosthetic shape (such as mandibular or postural defects caused by traumas, dental wear or incorrect interpretations of mandibular movements caused by the inevitable and unnatural craniofacial asymmetries). Devices of the known type do not meet these requirements.

### SUMMARY OF THE INVENTION

The aim of the present invention is to provide a device for recording craniofacial data of an individual by using a reference which is fixed and constant over time.

Within the scope of this aim, an object of the present invention is to provide a device for recording craniofacial data which allows to record, with a single application of the device, the right and left sagittal auriculo-orbital planes (Frankfort planes).

Another object of the present invention is to provide a device for recording craniofacial data which allows to record, with a single application of the device, any vertical asymmetries of the infraorbital points and horizontal movements thereof on the sagittal plane.

A further object of the present invention is to provide a device for recording craniofacial data which allows to record the auriculonasal plane (Camper's plane) with a single application of the device.

A still further object of the present invention is to provide a device for recording craniofacial data which allows to determine, with a single application of the device, the frontal and horizontal sagittal position of the condyles and of the glenoid cavities (cranial and mandibular asymmetries).

Another object of the present invention is to provide a device for recording craniofacial data which allows to determine measurements which allow to calculate the individual characteristic angles between the upper occlusal plane and any other characteristic bilateral plane with respect to a fixed universal plane such as the ground plane.

Another object of the present invention is to provide a device for recording craniofacial data which allows to measure asymmetries and angles referred to any significant point that is present on both cranial sides.

Another object of the present invention is to provide a device for recording craniofacial data which allows to measure the asymmetry between the nasal spine and the axis of the upper incisors.

Another object of the present invention is to provide a device for recording craniofacial data which is highly reliable, relatively simple to manufacture and at competitive costs.

This aim, these objects and others which will become better apparent hereinafter are achieved by a device for recording craniofacial data, particularly for determining facial asymmetries, characterized in that it comprises a plate-like element to which a supporting element for positioning the device in the mouth of a patient is connected in a fixed manner, said plate-like element being provided with a plurality of arms whose position is adjustable, said arms being suitable to support measurement means which are arranged on a vertical plane with respect to the plane on which said plate-like element is formed, means for defining a reference plane being provided in order to refer the measurements recorded by said measurement means to said reference plane.

### BRIEF DESCRIPTION OF THE DRAWINGS

Further characteristics and advantages of the present invention will become better apparent from the description of a preferred but not exclusive embodiment of the device according to the invention, illustrated only by way of non-limitative example in the accompanying drawings, wherein:

FIG. 1 is a perspective view of the cranium of an individual with the various planes that intersect it at right angles to each other;

FIG. 2 is a perspective view of the device according to the present invention, illustrating a supporting frame applicable to the device, shown in exploded view;

FIG. 3 is a perspective view of a detail of the device according to the invention;

FIG. 4 is a view of the method for measuring a frontal asymmetry by means of the device according to the present invention;

FIG. 5 is a view of a detail for locking the frame to the device according to the present invention; and

FIG. 6 is a plan view of the rules used to record Camper's plane and the Frankfort planes in the device according to the invention.

### DESCRIPTION OF THE PREFERRED EMBODIMENTS

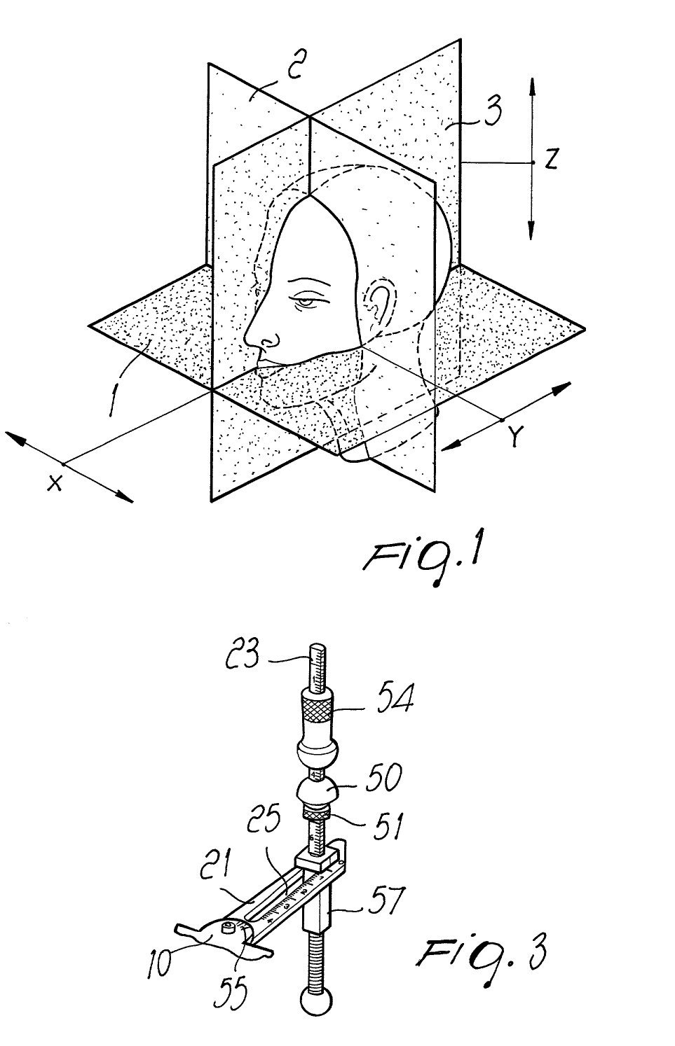

With reference to the above cited figures, FIG. 1 is a view of the cranium of a subject with the planes that intersect it at right angles to each other.

In particular, the reference numeral 1 designates a horizontal plane defined by two axes X and Y, the reference numeral 2 designates a front plane identified by a vertical axis Z and by the axis X, and the reference numeral 3 designates a sagittal plane defined by the vertical axis Z and by the axis Y.

FIG. 2 is an exploded perspective view of the device according to the present invention, which also illustrates the axes X, Y and Z defined above in FIG. 1.

The device according to the invention, generally designated by the reference numeral 100, comprises a plate-like element 10 to which a support 11 is fixed in a central position; said support is generally of the bite fork-like type and is meant to fix the upper occlusal block, required for the stabilization of the device with respect to the upper arch of a subject.

Slotted guides 12, 13 and 14 are formed in the plate-like element 10, respectively at the ends and centrally with respect to the plate-like element 10. The guides 12 and 14 allow the sliding and/or rotary engagement of a first pair of arms 15 and 16, which respectively support at least one vertical rule 17 and 18 each. The plate-like element 10 furthermore supports a second pair of arms 20 and 21, which in turn support a second pair of rules 22 and 23 which are also of the vertical type.

The rules 17, 18 and the rules 22 and 23 can slide along their respective arms in guiding slots, designated by 24 and 25 respectively for the first pair of arms and for the second pair of arms.

The position of the arms 15 and 16 in the respective guides 12 and 14 is indicated by graduated scales, and likewise the position of the vertical rules 17, 18 and 22, 23 is determined by graduated scales formed along the respective arms parallel to the guiding slots 24 and 25.

A third pair of rules 26 and 27 is arranged vertically in a guiding slot 28 which is formed centrally with respect to the plate-like element 10 and faces the fork-like supporting element 11.

The third pair of rules also comprises graduated rules 26 and 27, and an additional central rule 29 can slide in the guiding slot 28 in which the third pair of rules 26 and 27 slides.

The plate-like element 10 furthermore comprises a centering device 30, which can slide in the central guiding slot 13 and allows to record, on a dedicated scale, the asymmetry between the axis of the upper incisors and the facial axis recorded on the nasal spine.

One of the particularities of the invention is the fact that in order to render the measurement data universally comparable, a universal plane commonly termed “ground plane” is defined; all the measurements and therefore the inclinations recorded by said vertical rules can be transferred on the basis of said plane.

The universal plane is defined by vertical tubular elements, arranged along the axis Z, which are respectively designated by the reference numerals 31 and 32, for a first pair of tubular elements arranged so that they are rigidly coupled to the arm 15, and 33 and 34 for a second pair of tubular elements arranged so that they are rigidly coupled to the arm 16.

The tubular elements are conveniently transparent and are mutually connected by a tube 36, which allows their mutual connection. The tubular elements 31, 32, 33 and 34, as well as the tube 36, are filled by a liquid (more or less intensely colored water, diluted and colored alcohol, et cetera), whose free surface always arranges itself at the same level according to the principle of communicating vessels.

The hermetic closure of the tubes prevents any transfer of liquid from one vessel to another and therefore prevents any variation in level, thus defining a universal plane known as “ground plane”.

Instead of leveling with water columns (communicating vessels V), it is possible to use leveling by means of air bubbles arranged along the directrices X and Y and therefore at right angles to each other. As an alternative, or as an addition, it is possible to use automatic devices for finding the ground plane, such as laser beams trained on the level references, and the like.

Once the device according to the invention has been removed from the patient, it is sufficient to reposition the ground plane to have exactly the measured craniofacial positions and, if the measurements were performed with the patient in a static and erect habitual posture, the exact inclination assumed by the cranium with respect to the cervical vertebrae.

All the recorded points are referred to the scales dedicated to the individual movements on the conventional horizontal and vertical planes.

It is furthermore possible to plot the recordings on a chart in order to transmit the characteristic gnatho-craniofacial data to the prosthetics laboratory or for clinical records filing.

The device according to the invention also serves to preset the plaster-of-Paris bases on which it is possible to mark all the asymmetries with respect to the three fundamental planes 1-3 and to any planes different from the fundamental ones but nonetheless useful for anatomical studies.

This is achieved by means of a frame 40 in which there is, in a guided and centered manner, a base 41 onto which a casting of plaster of Paris, resin et cetera is performed.

Guides 42, 43, 44 and 39 are formed in the frame 40 and allow the sliding or rotation of two pairs of arms 45, 46 and 47, 48 which are entirely similar to the ones applied to the plate-like element 10 of the device and are also provided with identical graduated scales.

The movements of the arms 45-48 must duplicate exactly the movements of the arms 15, 16 and 20, 21 and are meant to perfectly center the axes X of the pairs of lateral rules 17, 18 and 22, 23.

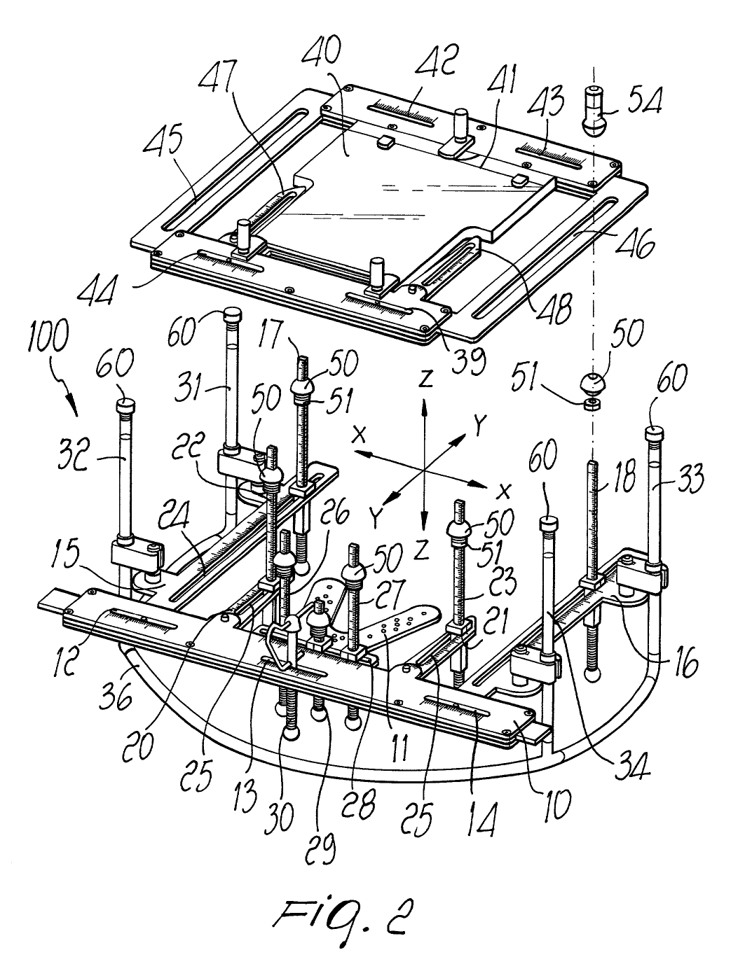

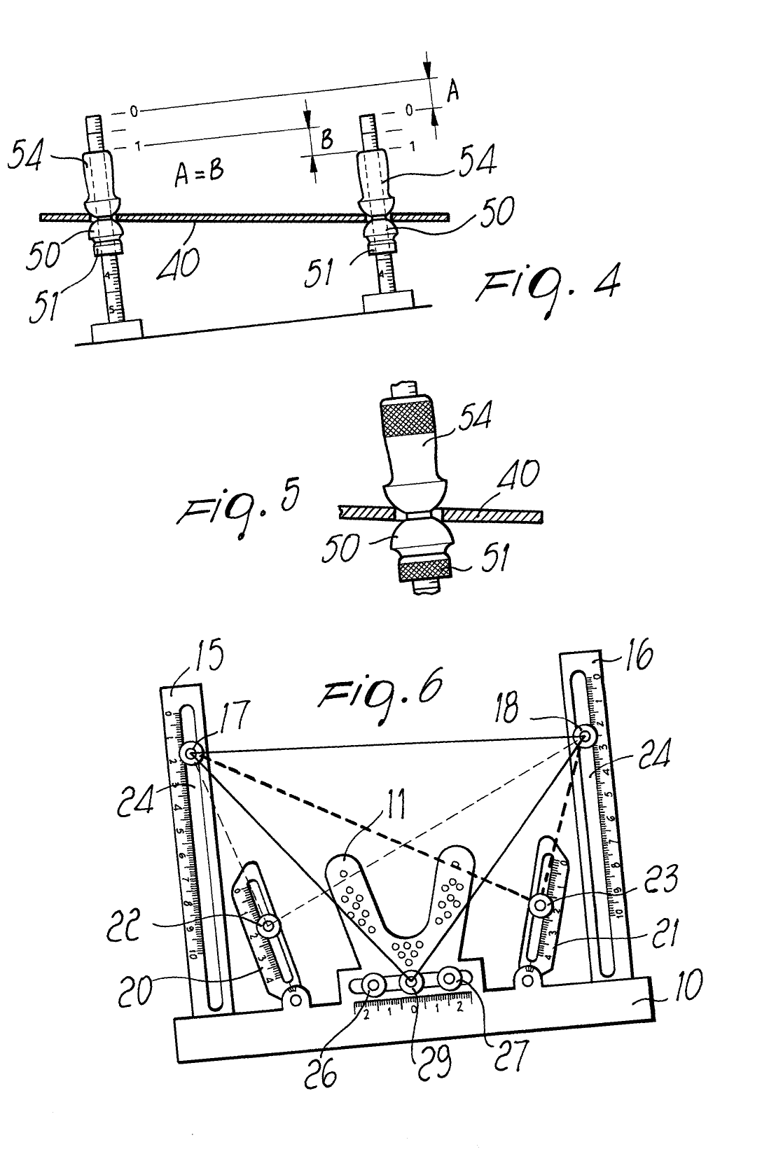

Nuts 50 with a spherical head are screwed onto the rules (all by the same amount or measurement with respect to the zero reference that corresponds to the upper plane of the rules or to the light ray emission point) and are locked in position by virtue of appropriate lock nuts 51, as shown in detail in FIG. 3.

The movement of the horizontal plane is meant exclusively to reduce the thickness of the plaster-of-Paris base while complying with the differences in level of the recorded asymmetries.

The frame 40 is then mounted on the lower vertical rules 17, 18 and 22, 23 by means of hemispherical lock nuts which are meant to lock the frame, which assumes positions, with respect to the horizontal plane, which are always defined by three resting points, as shown in FIG. 6.

The portion of plate-like element 10 that allows to support the arms 20 and 21 is provided with a goniometric scale 55 which allows to determine the angle of the respective arms 20 and 21.

The vertical rule 22, 23 is adjusted in the respective guide 25 by means of a lock nut 57.

In order to define Camper's plane, the Frankfort plane, and the planes studied for pathologic anatomy analysis, the rests on the lower nuts 50 that are not directly affected (nuts which trace the positions of the X axes) are excluded and the upper frame 40 is locked only by means of the nuts 50 affected by the plane being considered, which is in any case always acquired by triangulation, as shown in FIG. 6.

It is important to stress that after recording one plane it is always possible to restore the height referred to the Z axes by means of the graduated scales provided on the various rules.

With reference to the above cited figures, the operation of the device according to the invention is as follows.

The characteristic craniofacial planes are referred to the occlusal resting of the upper maxillary bone. The position of the device is obtained by means of the bite fork-like supporting device 11, on which an upper occlusal block (made for example of resin, thermoplastic material or the like) is centered and fixed.

After resting firmly the occlusal block against the upper arch of the subject, the position is maintained by virtue of conventional fixing methods, such as for example straps, helmet, temporary lower occlusal block, et cetera.

At this point the actual recording of the spatial position, along the three fundamental geometric axes X-Y-Z, of the craniofacial characteristic points of the right and left sides begins.

The recordings are performed by means of the rules which slide or rotate on the horizontal plane and have micrometric adjustment on the vertical plane (Z axis).

Each movement is performed on graduated scales with which the individual rules are provided. It is important to note that:

the measurements related to the X axis (which by convention is assumed to lie on the horizontal plane 1) are referred to the nasal spine (i.e. to the axis of the face) and provide measurements related to asymmetries on the frontal plane 2;

the measurements related to the Y axis (which also by convention is assumed to lie on the horizontal plane 1) provide values related to asymmetries on the sagittal plane 3;

the measurements related to the Z axis (which by convention is assumed to be the vertical plane) provide values related to asymmetries with respect to the horizontal plane.

Once the device according to the invention has been fixed as described above, the steps required to perform the spatial recordings of the cranium consist in:

checking the centering of the nasal spine (on the X axis);

positioning along the X-Y directrices the individual rules, making them correspond to the characteristic points being recorded and examined;

locking the X and Y positions of each rule by virtue of provided stop elements (nut 57) and adjusting the height of the individual rules along the Z axis, making the ends of the rules coincide with the characteristic points being acquired and examined;

and locking the positions with the provided stop elements or sending the reached position to an electronic data acquisition instrument.

Finally, while the subject being examined is in an erect static normal posture, the plugs 60 are loosened so as to close hermetically the tubular elements 31-34 and then closed when said tubular elements have reached stabilized levels.

If the device is provided with air-bubble levels, it is necessary to adjust leveling or find for the ground plane if the instrument is provided with automatic finding devices.

This last operation sets a universal planar reference such as the ground plane to which reference can be made at any time for gnathologic and postural studies of the subject. Finally, the device is removed from the subject being examined.

In practice, as shown for example in FIG. 6, in order to define Camper's plane and the Frankfort plane, the rests on nuts 50 that are not directly affected are excluded and the upper frame 40 is locked only by means of the nuts 50 affected by the plane being considered, which is taken always and in any case by triangulation.

FIG. 6 illustrates the principle of triangulation used to define Camper's plane, which is defined by excluding the rules 22, 23, 26 and 27 and by using only the rules 17, 18 and 29, thus performing a triangulation as shown in thick dashed lines in the figure.

In order to define the left Frankfort plane, the rules 29, 23, 26 and 27 are excluded and the rules 22, 18 and 17 are used instead.

The left Frankfort plane is therefore indicated by the thin dashed lines in FIG. 6.

The thick dashed lines, again in FIG. 6, instead indicate the triangulation used to define the right Frankfort plane, in which the rules 23, 17 and 18 are used, thus excluding the rules 29, 22, 26 and 27.

The various rules have the following meanings:

The rule designated by the reference numeral 29 indicates the nasal spine reference; the rules 17 and 18 provide auricular references; the rule 22 provides a left infraorbital reference; and the rule 23 provides a right infraorbital reference.

Finally, the rules 26 and 27 provide references for additional significant points.

Clearly, what has been described above is merely an example and all the rules can be used for anatomical recordings other than those mentioned above.

In practice it has been found that the device according to the invention fully achieves the intended aim and objects, since it allows to perform craniofacial measurements by using a constant reference plane as ground plane, by virtue of means suitable to form a fixed reference which are constituted by the tubular elements filled for example with liquid, so as to define a universal plane to which all the measurements and inclinations recorded by the rules can be traced.

The device thus conceived is susceptible of numerous modifications and variations, all of which are within the scope of the inventive concept. Thus, for example, the vertical spatial position of the rules (Z axis) can correspond to the position of the upper end of the rule at the level of the point to be recorded, or at a point which is lower than the end, if the centering of the characteristic cranial points is performed with light beams (for example carried to the spot by means of optical fibers, low-power laser beams, et cetera) concentrated on previously found and marked reference points (landmarks).

The spatial positions reached by the rules in the directrices X-Y-Z can be recorded in a direct visual manner, analogically, or be sent directly to an electronic data acquisition and processing unit.

The device according to the invention allows to perform, as a single device and in a single application, bilateral auriculo-orbital recordings (Frankfort planes), auriculonasal recordings (Camper's planes), bilateral recordings related to the hinge axis, recordings of the infraorbital line and any bilateral measurement deemed necessary for anatomical studies related to the spatial positioning of the characteristic individual cranial and gnathologic points and otherwise of points which are necessary for determining the asymmetries of cranial torsions with respect to the ground plane and to the centering of the face.

Furthermore, the device allows to faithfully replicate the spatial positions of the rules and therefore of the gnatho-cranio-postural characteristic points by relying exclusively on the ground plane and on the X-Y-Z spatial measurements traced according to the graduated scales dedicated to each individual rule.

Moreover, the device allows to record the asymmetries of the nasal spine with respect to the axis of the upper incisors and to produce directly on the same device “primitive” plaster-of-Paris bases, each of which records a characteristic plane (Frankfurt plane, Camper plane, or particular planes for anatomical studies) which allow comparative study of asymmetries.

All the details may furthermore be replaced with other technically equivalent elements.

In practice, the materials used, so long as they are compatible with the specific use, as well as the contingent shapes and dimensions, may be any according to requirements and to the state of the art.

The disclosures in Italian Patent Application No. MI2000A001995 from which this application claims priority are incorporated herein by reference.

What is claimed is:

1. A device for recording craniofacial data, particularly for determining facial asymmetries, comprising a plate-like element to which a supporting element for positioning the device in the mouth of a patient is connected in a fixed manner, said plate-like element being provided with a plurality of arms whose position is adjustable, said arms being suitable to support measurement means which are arranged on a vertical plane with respect to the plane on which said plate-like element is formed, means for defining a reference plane being provided in order to refer the measurements recorded by said measurement means to said reference plane.

2. The device according to claim 1, wherein said means for defining a reference plane comprise a plurality of tubular elements which are rigidly coupled to said plurality of arms connected to said plate-like element, said plurality of tubular elements being filled with a liquid and being closed hermetically, said plurality of tubular elements being mutually connected for a distribution of said liquid according to the principle of communicating vessels.

3. The device according to claim 1, wherein said plate-like element has guiding slots which allow the translational positioning of a first pair of said plurality of arms, a second pair of said plurality of arms being connected to said plate-like element so as to rotate.

4. The device according to claim 3, wherein each one of said plurality of arms is provided with guiding slots for the translational positioning of said measurement means.

5. The device according to claim 4, wherein said measurement means comprise graduated rods.

6. The device according to claim 5, wherein said graduated rods are fixed to said arms, within the respective guiding slots, by means of nuts.

7. The device according to claim 6, wherein said graduated rods are rules.

8. The device according to claim 1, comprising, at a central region of said plate-like element, a guiding slot for a centering device which is suitable to record an asymmetry between the axis of the upper incisors and the facial axis recorded on the nasal spine of the subject.

9. The device according to claim 1, wherein said supporting element suitable to be inserted in the mouth of the subject is arranged centrally with respect to said plate-like element.

10. The device according to claim 5, comprising an upper frame which is suitable to support a base element and can be connected rigidly to said graduated rods.

11. The device according to claim 10, wherein said frame comprises a first pair of arms and a second pair of arms which can be respectively arranged at said first and second pairs of arms which are connected to said plate-like element.

12. The device according to claim 11, wherein said first and second pairs of arms of said upper frame correspond to said first and second pairs of arms connected to said plate-like element, respectively.

13. The device according to claim 10, wherein said upper frame is connected to said plurality of graduated rods by means of a nut which has a hemispherical head and is suitable to abut against a nut with a corresponding lock nut having a hemispherical head which is screwed onto each one of said measurement rods.

14. The device according to claim 11, wherein said first and second pairs of arms of said upper frame are each provided with a slot suitable to allow the insertion of a respective said measurement rod.

15. The device according to claim 1, wherein said measurement means comprise transducers.

16. The device according to claim 1, wherein said means for defining a reference plane comprise air-bubble leveling means.
